(19) United States
(12) Patent Application Publication
(10) Pub. No.: US 2002/0111531 A1
(43) Pub. Date: Aug. 15, 2002

(54) Surfactant enhanced embryo transfer

(76) Inventors: Ghanima Maassarani, Las Vegas, NV (US); Geoffrey Sher, Las Vegas, NV (US)

Correspondence Address: BAZERMAN & DRANGEL, P.C., 60 EAST 42ND STREET, SUITE 820, NEW YORK, NY, 10165, US

(21) Appl. No.: 10/063,431

(22) Filed: Apr. 23, 2002

Related U.S. Application Data
(60) Provisional application No. 60/285,389, filed on Apr. 23, 2001.

(51) Int. Cl. A61B 17/43

(52) U.S. Cl. 600/35; 600/33

(57) ABSTRACT

An enhanced method of in vitro fertilization by coating an embryo transfer catheter with a non-embryotoxic phospholipid. The phospholipid reduces or eliminates the collection of mucus on an ET catheter as it passes through the mucus plug in the cervical cannel and thus elimininating its detrimental effect in, engulfing the embryo and preventing implantation.

### BACKGROUND OF INVENTION

1. Field of the Invention

This invention relates to a method for introducing and implanting an embryo into the uterus.

2. Prior Art

Human In Vitro Fertilization (IVF) is the extracorporeal (outside the body) fertilization of a human egg thereby producing an embryo. To optimize pregnancy potential, the fertilized egg(s), i.e., embryo(s) must be accurately and smoothly placed in the uterine (endometrial) cavity and then retained in that location. Typically one or more embryos are transferred into the uterine cavity after culturing for two to six days. The process of placing the embryo(s) is called Embryo Transfer (ET).

An embryo is normally transferred into the uterus by means of a polytetrafluoroethylene or Teflon® catheter. The embryo is suspended in a fluid medium in the catheter. The catheter is passed through the cervical opening, via the cervical canal into the endometrial cavity. Conventional means such as ultrasound and fiber optics may be used to guide and position the catheter in the uterus. Upon entrance into and proper placement in the cavity, the fluid containing the embryo is flushed through the catheter and the embryo is carried into the uterus.

Less than one third of the embryos so transferred result in a live birth. A successful outcome following the in vitro fertilization and embryo transfer is in a large part dependent upon the appropriate placement of the transferred embryo in the uterine cavity. Descent or reflux of transferred embryos into the cervical canal during withdrawal of the ET catheter results in their sequestration and renders such embryos unavailable for implantation.

Attempts have been made to reduce the incidence of embryo reflux by a number of methods including:

1. Use of a flexible ET catheter to avoid inducing uterine irritability.

2. Use of fibrin as a sealant (glue) to “attach” the transferred embryos to the uterine wall.

3. Careful measurement of the uterine depth and ultrasound and/or fiberoptic guidance to promote accurate placement of embryos in the uterine cavity.

4. Use of uterine tocolytics such as β-adrenergic and anti-prostaglandin agents to relax the uterus in preparation for ET.

For example, U.S. Pat. No. 6,010,448, to Thompson, issued on Jan. 4, 2000 discloses the use of an adhesive carried by the distal end of the catheter to glue the embryo to the uterine wall and a balloon to position and hold the embryo in place against the uterine wall.

However, any one, or the combination, of these techniques fail to sufficiently increase the pregnancy rate. The present invention relates to a method for promoting proper embryo placement and retention, thereby increasing the likelihood of the embryo(s) being available for subsequent implantation into the uterine lining (endometrium), further reducing embryo reflux.

### SUMMARY OF INVENTION

We have found that the interaction of the catheter with the mucus plug in the cervical canal (the entrance to the uterus) is a major cause of IVF/ET failure. The cervical mucus adheres to the transferred catheter and is carried into the uterus during insertion. This not only leads to inadvertent introduction of bacterial and cellular debris into the normally sterile endometrial cavity, but mucus at the catheter tip may also engulf the embryo, diminishing its ability to implant or act to adhere the embryo to the catheter causing it to be pulled down into the cervical canal and /or vagina as the catheter is removed. Thus, in many cases, implantation failure is due to embryo sequestration within the cervical mucus.

The present invention is designed to overcome this effect of the mucus plug on the ET transfer through the use of phospholipid, a non-embryotoxic surface tension lowering substance, as a coating on the catheter. In accordance with the present invention, the embryo is protected from the mucus plug and the likelihood of the embryo being actually removed from the uterus upon withdrawal of the ET catheter is reduced by the application of a the phospholipid, Phosphatidylcholine, to the exterior surfaces of the ET catheter and its outer sheath (canula). The phospholipid lowers the surface tension on and at and in the tip of the catheter. Such reduction in surface tension improves the easy passage of the ET catheter through the cervical canal, minimizes the risk of introducing mucus and cellular debris into the uterine cavity, promotes proper discharge of the embryo(s) into the uterine cavity by reducing the likelihood of an embryo adhering to the catheter, and thereby enhances embryo availability for implantation. Naturally occurring phospholipids in general and, phosphatidylcholine in specific, are not harmful to (and may even enhance) embryo development and/or implantation. Some Examples of other acceptable phospholipids for such use are: phosphatydil glycerol, sphingomyelin and phosphatydil ethanolamine.

Immediately before use, the surfactant phospholipid is liberally applied both to the outer surfaces of the ET catheter and its sheath (canula). Such application has a number of positive effects including elimination of an embryo's attachment to the outer surface of the ET catheter, reducing the likelihood of the embryo-containing culture medium sticking to the ET catheter tip and being drawn back into the cervical canal at the time of the catheter withdrawal. As a result, there is improved uterine retention of transferred embryos and higher embryo implantation (pregnancy) rates following ET.

### DETAILED DESCRIPTION

The present invention is an improvement of embryo transfer technology to eliminate the adverse effects of the ET catheter passing through the cervical canal and its mucus plug. When the ET catheter passes through the mucus plug, it is often coated, in at least part, with mucus including mucus at and near the tip of the catheter. Such mucus and other “debris”, derived from the cervix may act as a barrier to the correct implantation of the embryo(s) into the endometrium, by 1) causing their adherence to the catheter and thus allowing the embryo(s) to be dragged into the cervical canal when the catheter is removed and, 2) by acting as a physical barrier that interfaces between the embryo and the endometrial lining, thus interfering with attachment (implantation) of the embryo(s) to the endometrium. In the present invention, a non-embryotoxic phospholipid surfactant acts as a surface tension lowering agent, which coats the ET catheter and canula. The surfactant acts to lessen the accumulation of mucus and other “debris” on the catheter as it passes through the cervical canal, into the uterus and lessens the ability of any mucus, which does remain on the catheter from causing the embryo(s) to stick to the catheter. One acceptable surfactant phospholipid is presently offered by Abbott Laboratories under the trademark SURVANTA. The Survanta is a solution containing a natural bovine lung extract comprising 25 mg/ml of phospholipids, with 11-15 mg/ml of phosphatidylcholine suspended in 0.9% saline. It is normally vaporized and sprayed into the lungs of neonates with Respiratory Distress Syndrome (Hyaline membrane disease). Mouse embryos toxicity assays found a 1:3 dilution of Survanta with Human tubular fluid to have no adverse effect on fertilization, embryo development and blastocyst formation.

The present invention can be used with any ET catheter. The sterile catheter may be flushed with a diluted phospholipid prior to loading the embryo(s). Immediately prior to the ET, a phospholipid is liberally applied along the distal 5 cm of the outer surface of both the ET catheter and its sheath (canula). Such application of human tubal fluid prevents cervical mucus, blood and cellular debris from adhering to the ET catheter, thereby reducing the possibility of embryo retention. Given this coating, an embryo is less likely to be blocked on its passage from the catheter to the uterine wall and less likely to adhere to the exterior of the catheter and thus be dragged out of position with the catheter's removal.

The invention is further illustrated by the following example:

### EXAMPLE 1

A group of 32 women under 39 years of age, with a normal uterine cavity and normal ovarian reserve, were stimulated for IVF/ET standard protocols. Embryos were cultured in P1 (Irvine Scientific embryo growth media) until day 3. Some embryos were then allowed to continue in culture in a blastocyst medium (Irvine Scientific) for an additional 2-3 days. Embryo transfer was performed on day 3, 5 or 6, Embryo transfer was performed using a Wallace catheter under ultrasound-guidance after flushing and cleansing the cervical canal with culture media. The patients were randomized to receive standard ET (Group A, n=15) or ET in accordance with the present invention (Group B, n=17). After the embryo(s) were drawn up into the distal end of the catheter the ET catheters in Group B were coated with dilute 1:3 Survanta to the outer surfaces of the distal 5 cm of both the catheter and canula. ET was performed within 2 minutes thereof thereafter under abdominal ultrasound guidance.

Each woman received between one and 3 embryos during a single procedure session. The woman of Group A received a total of 40 embryos transferred and Group B received a total of 49 embryos transferred. In Group A, there were 13 embryos successfully implanted, (i.e., viable conceptii confirmed by ultrasound examination after the twelfth week of pregnancy) in 9 women while in Group B, there were 27 embryos successfully implanted in 12 women. Thus, the implantation rate of successful embryo transfers for Group A was 33% and Group B was 55% percent. Several woman were carrying more than one child as it is common to transfer multiple embryos during implantation. As seen from the data, the rate of successful implantation was substantially higher per embryo transfer in Group B.

While the invention has been described in detail and with reference to the specific embodiment thereof, it will be apparent that various changes and modifications can be made therein without varying from the spirit and scope thereof, such as the use of alternative non-embryo toxic human tubal fluid.

1. A method of implantation of an embryo by a catheter into a pre-selected optimum site on the endometrium of a female uterus, comprising the steps of:
providing a catheter for use in said uterus;
loading the catheter with an embryo;
coating said catheter with a non-embryotoxic surfactant; and
depositing said embryo into said endometrium.

2. The method of claim 1 in which the catheter is flushed with a solution of the non-embryotoxic surfactant prior to loading the catheter with the embryo.

3. The method according to claim 1 where the non-embryotoxic surfactant is comprised of a compound containing a phospholipid.

4. The method according to claim 3 where the phospholipid is a phosphatidycholine.

5. The method according to claim 3 where the phospholipid is a phosphatydil glycerol.

6. The method according to claim 3 where the phospholipid is a sphingomyelin.

7. The method according to claim 3 where the phospholipid is a phosphatydil ethanolamine.
